(19) United States
(12) Patent Application Publication
(10) Pub. No.: US 2016/0022604 A1
(43) Pub. Date: Jan. 28, 2016

(54) DIRECTLY COMPRESSED OSPEMIFENE COMPOSITIONS

(71) Applicant: CADILA HEALTHCARE LIMITED, Ahmedabad (IN)

(72) Inventors: Sushrut Krishnaji Kulkarni, Ahmedabad (IN); Pavak Rajnikant Mehta, Ahmedabad (IN); Ritesh Kapoor, Ahmedabad (IN)

(73) Assignee: CADILA HEALTHCARE LIMITED, Ahmedabad (IN)

(21) Appl. No.: 14/805,733

(22) Filed: Jul. 22, 2015

(30) Foreign Application Priority Data
Jul. 23, 2014 (IN) 2376/MUM/2014

(51) Int. Cl. A61K 31/085; A61K 9/20; A61K 9/14

(52) U.S. Cl. CPC A61K 31/085; A61K 9/14; A61K 9/2095

(57) ABSTRACT

The present invention relates to pharmaceutical compositions comprising ospemifene or a pharmaceutically acceptable salt thereof prepared by direct compression process. The compositions of the invention may be advantageously used for the treatment or prevention of atrophy-related diseases or disorders in women, especially in women during or after the menopause.

### FIELD OF THE INVENTION

The present invention relates to pharmaceutical compositions comprising ospemifene or a pharmaceutically acceptable salt thereof prepared by direct compression process. The compositions of the invention may be advantageously used for the treatment or prevention of atrophy-related diseases or disorders in women, especially in women during or after the menopause.

### BACKGROUND OF THE INVENTION

Ospemifene is known as 2-[4-(4-chloro-1,2-diphenylbut-1-enyl)phenoxy]ethanol and it is the Z-isomer of the compound of following structural formula:

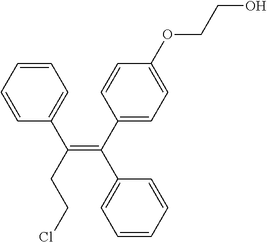 [SMILES: OCCOc1ccc(/C(=C(/CCCl)c2ccccc2)c2ccccc2)cc1]

It is one of the main metabolites of toremifene, is known to be an estrogen agonist and antagonist as described in Kangas, 1990; PCT publication Nos. WO 1996/07402 and WO 1997/32574. The compound is also called (deaminohydroxy) toremifene and it is also known under the code FC-1271a. Ospemifene has relatively weak estrogenic and antiestrogenic effects in the classical hormonal tests as described in Kangas, 1990.

According to PCT publication Nos. WO 1996/07402 and WO 1997/32574, ospemifene has anti-osteoporosis actions and it decreases total and LDL cholesterol levels in both experimental models and in human volunteers.

The use of ospemifene, a selective estrogen receptor modulator (SERM) for the treatment of certain climacteric disorders in postmenopausal women, namely vaginal dryness and sexual dysfunction, is disclosed in PCT publication No. WO 2002/07718.

Ospemifene belongs to class-II category of the biopharmaceutical classification system. The compound is highly lipophilic and its relatively low aqueous solubility makes it difficult to provide a dosage form. Therefore, more soluble and bioavailable compositions of ospemifene are needed which may be prepared by non-tedious techniques. There is still a need for an alternate ospemifene composition which could ease and simplify the overall formulation process.

PCT publication No. WO 2005/079777 relates to solid drug composition comprising granulates containing a therapeutically active compound, which is ospemifene. The application discloses the formulation of ospemifene using granulation technique advantageously over direct compression process. The specification discloses the tablet containing granulates significantly improves the dissolution of ospemifene, compared to tablets manufactured by direct compression and the conventionally tableted (directly compressed) ospemifene failed to dissolve completely even after three hours. At 240 minutes, conventionally tableted ospemifene was only 80% dissolved while the granulated ospemifene tablet was 80% dissolved at thirty minutes and nearly completely dissolved in 120 minutes.

The current invention provides a novel immediate release composition of ospemifene for oral administration wherein the composition is prepared by a direct compression process and releases about 80% of ospemifene or a pharmaceutically acceptable salt thereof in about 30 minutes.

### SUMMARY OF THE INVENTION

In a general aspect, there is provided an oral pharmaceutical composition comprising:

a) ospemifene or a pharmaceutically acceptable salt thereof; and
b) one or more pharmaceutically acceptable excipients,
wherein the composition is devoid of any granulates and releases at least about 80% of ospemifene or a pharmaceutically acceptable salt thereof in about 30 minutes.

In another general aspect, there is provided a pharmaceutical composition, wherein the composition is prepared by a direct compression process.

In another general aspect, there is provided a pharmaceutical composition, wherein the composition comprises about 20 mg to about 100 mg of ospemifene, more preferably about 30 mg to about 90 mg of ospemifene.

In another general aspect, there is provided a pharmaceutical composition of ospemifene, wherein the composition is in the form a tablet, a capsule, pellets or powder.

In another general aspect, there is provided a pharmaceutical composition, wherein 50% of ospemifene has a particle size of not more than about 15 microns.

Embodiments of the pharmaceutical composition may include one or more of the following features. For example, the pharmaceutical composition may further comprise one or more pharmaceutically acceptable excipients. The pharmaceutically acceptable excipients may comprise one or more binders, fillers, disintegrants, lubricants, glidants, sweetening agents, anti-tacking agents and the like.

In yet another general aspect, there is provided a pharmaceutical composition of ospemifene further comprising one or more enhancing agents.

In yet another general aspect, there is provided a pharmaceutical composition of ospemifene comprising one or more enhancing agents, wherein the enhancing agent comprises one or more solubility enhancing agents, dissolution enhancing agents, absorption enhancing agents, penetration enhancing agents, surface active agents, stabilizing agents or combinations thereof.

In another general aspect, there is provided a process for preparing a pharmaceutical composition, wherein the process comprises the steps of:

(a) mixing ospemifene with one or more enhancing agents and optionally one or more pharmaceutically acceptable excipients,
(b) blending the mixture,
(c) compressing the resulting blend into tablets; and
(d) optionally coating the tablets.

Embodiments of the pharmaceutical composition may include one or more of the following features. For example, the pharmaceutical composition may further comprise one or more pharmaceutically acceptable excipients. The pharmaceutically acceptable excipients may comprise one or more binders, fillers, disintegrants, lubricants, glidants, anti-tacking agents and the like.

In another general aspect, there is provided a method of treatment or prevention of atrophy-related diseases or disorders in women, especially in women during or after the menopause, comprising orally administering to the subject a therapeutically effective amount of the composition of ospemifene as per the invention.

The details of one or more embodiments of the invention are set forth in the description below. Other features of the invention will be apparent from the description.

### DETAILED DESCRIPTION OF THE INVENTION

It has now surprisingly been found that ospemifene compositions can be prepared, wherein 50% of ospemifene has a particle size of not more than about 15 microns and wherein at least about 80% of the composition is dissolved within 30 minutes after subjecting said composition to dissolution testing. The tablets of the present invention, produced by direct compression technique significantly improves the dissolution of ospemifene, compared to the tablets described in the art produced using the same direct compression technique. Also, ospemifene compositions of the present invention show almost complete ospemifene dissolution within 60 minutes of the start of dissolution testing i.e. directly compressed ospemifene was at least 90% dissolved at 60 minutes and at least 95% dissolved in 120 minutes.

Granulation is a process where primary powder particles are made to adhere to form larger, multiparticle entities called granules. Pharmaceutical granules typically have a size range between 0.3 and 4.0 mm, depending on their subsequent use. Granules (also referred herein as granulates) are typically produced as an intermediate product for the production of tablets or capsules. However, problems often associated with granulation techniques include choice and method of addition of the binder and the effect of drying time and temperature on drug stability and its distribution within the solid mass.

In the compositions of the present invention, direct compression has been advantageously used as it provides the shortest, most effective and least complex way to produce tablets. The compositions of the invention are thus devoid of granulates and are prepared by techniques which need less equipment and incorporate less number of stages in the overall composition process, and thus will be simple and economically viable.

The term “ospemifene” includes a geometric isomer, a polymorph, a stereoisomer, a pharmaceutically acceptable salt, an ester thereof or a metabolite thereof.

The term “particle size” refers to the particle diameter, or in case the particles are not spherical, to the largest extension in one direction of the particle.

The term “immediate release composition” refers to a composition that releases greater than or equal to about 80% of ospemifene in less than or equal to about 1 hour.

The term “almost complete dissolution” refers to the phenomenon where more than 90% of ospemifene is dissolved.

For the purposes of this application, an “enhancing agent” (an enhancer), is defined as any pharmaceutically inactive ingredient that improves the efficacy and therapeutic potential of a composition.

The composition according to the present invention comprises ospemifene or a pharmaceutically acceptable salt thereof, wherein 50% of ospemifene has a particle size of not more than about 15 microns (D50), preferably not more than about 10 microns and 90% of ospemifene has a particle size of not more than about 20 microns (D90), preferably not more than about 15 microns.

In one embodiment, the ospemifene tablets of the invention preferably have a hardness of about 10-180N, particularly preferably about 40-140N.

In another embodiment, the ospemifene tablets of the invention preferably have a friability of less than 3%, particularly preferably less than 2%, especially less than 1%.

In yet another embodiment, the ospemifene tablets of the invention usually have a “content uniformity” of 90 to 110% of the average content, preferably 95 to 105% (This means that all the tablets have a content of active agent of between 90 and 110%, preferably between 95 and 105% of the average content of active agent).

The pharmaceutically acceptable excipients suitable for present invention comprise one or more of binders, fillers, disintegrants, lubricants, glidants, anti-tacking agents, plasticizers and the like.

Suitable enhancing agents for the purpose of the present invention may include one or more of solubility enhancing agents, dissolution enhancing agents, absorption enhancing agents, penetration enhancing agents, surface active agents and stabilizing agents. The representative, but non-limiting examples of these compounds are Vitamin E TPGS, amino acids such as glutamic acid and glycine, sorbitol, mannose, amylose, maltose, mannitol, lactose, sucrose, glucose, xylitose, dextrins such as maltodextrin, one or more of an anionic surfactant, a cationic surfactant or a non-ionic surfactants, for example Cremophor RH40 (glycerol-polyethylene glycol oxystearate), Gelucire 50/13 (PEG-32 glyceryl palmitostearate), sodium lauryl sulfate, Tween 80 (polyoxyethylene sorbitan monooleate also known as polysorbate 80), benzyl alcohol, Span 20 (sorbitan monolaurate), Poloxamer 407, polyethylene glycols, such as PEG3350; polyvinylpyrrolidones such as PVP K25, polyvinylalcohols, polyalcohols, oleic acid, Capmul GMO (glyceryl monooleate), sodium benzoate, cetyl alcohol, crospovidone, sodium starch glycolate, croscarmellose sodium, carboxymethylcellulose, starch, pregelatinized starch, HPMC, substituted hydroxypropyl cellulose, sodium bicarbonate, calcium citrate, sodium docusate, and menthol, among others.

Polysorbate 80, which is also known as Tween 80 is a non-ionic surfactant and emulsifier derived from polyethoxylated sorbitan and oleic acid. It is available in liquid as well as dry form. Enhancers may be combined to achieve multiple enhancement effects, for example, solubility enhancement combined with permeability enhancement, or to provide a synergistic effect to achieve greater and more efficient enhancement.

Suitable binders may include one or more of carbomers, dextrin, ethyl cellulose, shellac, zein, gelatin, polymethacrylates (eg. eudragit), pregelatinized starch, sodium alginate, gums, synthetic resins, silicic acid, hydrophilic polymers and the like.

The term “hydrophilic polymer” may include polymers with polar groups. Examples of polar groups are hydroxy, amino, carboxy, carbonyl, ethers, esters, and sulfonates. Examples of suitable hydrophilic polymers are cellulose derivatives, in particular hydrophilic derivatives of the cellulose (e.g. hydroxypropyl methylcellulose (HPMC), hydroxypropyl cellulose (HPC), carboxymethylcellulose or their sodium or calcium salt, hydroxyethyl cellulose), polyvinylpyrrolidone, preferably with a molecular weight of from 10,000 to 60,000 g/mol, copolymers of PVP, preferably co-polymers comprising vinylpyrrolidone and vinylacetate units (e.g. povidone, VA64, BASF), preferably with a molecular weight between 40,000 and 70,000 g/mol, poly(oxyethylene)alkyl ether, polyethylene glycol, co-block polymers of ethylene oxide, and propylene oxide (poloxamer, pluronic), derivatives of polymethacrylates, polyvinyl alcohol, polyvinyl alcohol derivatives, polyethylene glycol, and polyethylene glycol derivatives.

Suitable fillers may include one or more of microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol, erythritol and the like. The filler may be present in an amount of 5 to 80% by weight of the composition.

Suitable disintegrants may include one or more of croscarmellose sodium, sodium starch glycolate, low substituted hydroxypropyl cellulose (L hydroxypropyl cellulose), pregelatinized starch, sodium carboxymethyl cellulose, cross-linked polyvinylpyrrolidone and the like. The disintegrating agent may be present in an amount ranging from 0.1% to 20% w/w of the composition.

Suitable lubricants and glidants may include one or more of talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and the like. It would be appreciated that a person skilled in the art is cognizant of the fact that lubricant, glidant or anti-tacking agent may be used interchangeably. The lubricant, glidant or anti-tacking agent may be present in an amount ranging from 0.1% to 15% w/w of the composition.

Suitable anti-tacking agents may include one or more of talc, magnesium stearate and the like.

Suitable plasticizers may include one or more of triacetin, diethyl phthalate, tributyl sebacate, polyethylene glycol and the like.

In one embodiment, the pharmaceutical compositions of the invention may comprise about 30 mg to 90 mg of ospemifene, an enhancing agent and one or more other pharmaceutically acceptable excipients, wherein 50% of ospemifene has a particle size of not more than about 15 microns.

In another embodiment, the pharmaceutical compositions of the invention may comprise ospemifene, wherein 50% of ospemifene has a particle size of not more than about 15 microns and where ospemifene may be milled with one or more pharmaceutically acceptable excipients, dried and may finally be compressed using suitable tooling.

The pharmaceutical compositions as described herein may be prepared by processes known to the person having ordinary skill in the art of pharmaceutical technology, particularly direct compression. The process may further comprise co-milling, homogenization, spray coating, and the like.

Milling is generally performed in conventional milling apparatuses, such as in a ball mill, air jet mill, pin mill, classifier mill, cross-beater mill, disk mill, mortar grinder, rotor mill. The milling time may be 0.5 minutes to 2 hours, preferably 2 minutes to 1 hour, more preferably 5 minutes to 30 minutes.

Exemplary homogenization method comprises dispersing particles of ospemifene with one or more pharmaceutically acceptable excipients, in a liquid dispersion medium, followed by subjecting the dispersion to homogenization to reduce the particle size of Ospemifene to the desired effective particle size.

Compression may be performed with tableting machines known in the state of the art. The compression is preferably performed in the absence of solvents.

Examples of suitable tableting machines are eccentric presses or rotary presses. In the case of rotary presses, a compressive force of 2 to 40 kN, preferably 2.5 to 35 kN, is preferably applied.

The current invention encompasses a method of preparing a pharmaceutical composition, wherein the process may comprise mixing ospemifene and one or more enhancing agents and optionally one or more pharmaceutically acceptable excipients. The mixing may be performed in conventional mixers.

The mixing may, for example, be performed in compulsory mixers or free-fall mixers.

In yet another embodiment, there is provided a method of preparing a pharmaceutical composition, wherein the process may comprise the steps of:

(a) mixing ospemifene with one or more enhancing agents and optionally one or more pharmaceutically acceptable excipients,
(b) blending the mixture,
(c) compressing the resulting mixture into tablets; and
(d) optionally coating the tablets.

In another embodiment, there is provided a method of preparing a pharmaceutical composition, wherein the process may comprise the steps of:

a) milling ospemifene with one or more enhancing agents,
b) mixing the milled particles with one or more pharmaceutically acceptable excipients,
c) blending the mixture obtained; and
d) processing the milled particles using suitable tooling to obtain the composition.

The composition of the present invention may be additionally coated with an over-coat. The over-coat may be a moisture barrier coat, a protection coat, a seal coat, a taste-masking coat, a flavor coat, a polish coat, a color coat, or any other cosmetic coat that does not interfere with the release of the active compound or the enhancing agent. Suitable coating materials for such an over-coat are known in the art, and include, but are not limited to, cellulosic polymers such as hydroxypropyl methylcellulose, hydroxypropyl cellulose and microcrystalline cellulose, or combinations thereof (for example various Opadry® coating materials).

The compositions according to the present invention is useful in any application of ospemifene, especially when the compound is used for treatment or prevention of osteoporosis or for treatment or prevention of symptoms related to skin atrophy, or to epithelial or mucosal atrophy.

A particular form of atrophy which can be inhibited by administering of ospemifene is urogenital atrophy. Symptoms related to urogenital atrophy can be divided in two subgroups: urinary symptoms and vaginal symptoms. As examples of urinary symptoms can be mentioned micturation disorders, dysuria, hematuria, urinary frequency, sensation of urgency, urinary tract infections, urinary tract inflammation, nocturia, urinary incontinence, urge incontinence and involuntary urinary leakage.

As examples of vaginal symptoms can be mentioned irritation, itching, burning, maladorous discharge, infection, leukorrhea, vulvar pruritus, feeling of pressure and postcoital bleeding.

In another embodiment, the ospemifene compositions of the present invention provide for a relative Cₘₐₓ in the range of 80% to 125%, as compared to the same amount of ospemifene administered as the currently marketed immediate release composition (OSPHENA®).

The invention is further illustrated by the following examples which are provided to be exemplary of the invention and do not limit the scope of the invention.

### Example 1

TABLE 1a

| | | |
|---|---|---|
| S.N | Ingredient | % w/w |

Part A

| | | |
|---|---|---|
| 1. | Ospemifene | 25.6 |
| 2. | Microcrystalline cellulose | 31.2 |
| 3. | Mannitol | 12.8 |
| 4. | Pregelatinized starch | 17.1 |
| 5. | Sodium starch glycolate | 5.1 |
| 6. | Povidone K-30 | 1.7 |
| 7. | Polysorbate 80 (powder) | 2.5 |
| 8. | Colloidal silica anhydrous | 1.2 |
| 9. | Magnesium stearate | 0.8 |

Part B (Coating)

| | | |
|---|---|---|
| 10. | Opary white | 1.71 |
| 11. | Purified water | q.s |

### Process:

Ospemifene (D50=about 3 microns; D90=about 6.7 microns), microcrystalline cellulose, mannitol, pregelatinized starch, sodium starch glycolate, povidone, polysorbate 80 (Sepitrap®), colloidal silica anhydrous and magnesium stearate were sifted separately. Ospemifene, polysorbate 80 and colloidal silica anhydrous were mixed and co-sifted. To this mixture, sodium starch glycolate, povidone k-30, mannitol and microcrystalline cellulose were blended and the mixture so obtained was co-sifted using an appropriate sieve. The mixture was further blended in a blender for about 5 to about 20 minutes. The blend was lubricated using magnesium stearate and compressed into tablets using an appropriate tooling. The tablets obtained were coated using a homogeneous solution/dispersion of opadry white in purified water. The tablets were dried and appropriately packaged.

### Dissolution Data for Example 1

The dissolution performance was measured using a USP-II paddle type apparatus. Release times were measured by allowing the tablet to sink to the bottom of the vessel. A small, loose piece of non-reactive material, such as not more than a few turns of wire helix, may be attached to dosage units that would otherwise float. The paddle blade is rotated at 50 rpm. Aliquots were withdrawn for up to 2 hours. The samples were filtered immediately and analyzed using a spectrophotometer.

TABLE 1b
Dissolution performance for the final formulation of Example 1

| | Time | % drug |
|---|---|---|
| Medium | (hour) | release |
| Purified water + 2.0% SDS | 5 | 33.4 |
| (sodium dodecyl sulphate), | 10 | 62.2 |
| 900 ml. | 20 | 85.2 |
| | 30 | 88.6 |
| | 45 | 93.5 |

The tablets produced had a hardness of about 102.9 to about 122.5N.

### Example 2

TABLE 2

| | | |
|---|---|---|
| S.N | Ingredient | % w/w |

Part A

| | | |
|---|---|---|
| 1. | Ospemifene | 20-30 |
| 2. | Lactose | 40-60 |
| 3. | Docusate sodium | 2-5 |
| 4. | Povidone | 3-6 |
| 5. | Sodium starch glycolate | 3-5 |
| 6. | Colloidal silicon dioxide | 0.5-3 |
| 7. | Magnesium stearate | 0.5-3 |

Part B

| | | |
|---|---|---|
| 8. | Opadry ® | 1-3 |
| 9. | Water | q.s |

### Process:

Ospemifene (D₅₀ not more than 15 microns), lactose, docusate sodium, povidone, sodium starch glycolate and colloidal silicon dioxide were sifted through an appropriate sieve and mixed. The mixture was blended in a conta-blender. Magnesium stearate was sifted through an appropriate sieve and added to the blended mixture. The mixture obtained was again blended in a conta-blender. The blend was compressed using a suitable tooling to obtain tablets. The tablets were then coated with a dispersion of Opadry® in water.

### Example 3

TABLE 3

| | | |
|---|---|---|
| S.N | Ingredient | % w/w |

Part A

| | | |
|---|---|---|
| 1. | Ospemifene | 20-30 |
| 2. | Mannitol | 40-60 |
| 3. | Poloxamer | 2-5 |
| 4. | Hydroxypropyl methylcellulose | 3-6 |
| 5. | Crospovidone | 3-5 |
| 6. | Talc | 0.5-3 |
| 7. | Magnesium stearate | 0.5-3 |

Part B

| | | |
|---|---|---|
| 8. | Opadry ® | 1-3 |
| 9. | Water | q.s |

### Process:

Ospemifene (D₅₀ not more than 15 microns), mannitol, poloxamer, hydroxypropyl methylcellulose, crospovidone and talc were sifted through an appropriate sieve and mixed. The mixture was blended in a conta-blender. Magnesium stearate was sifted through an appropriate sieve and added to the blended mixture. The mixture obtained was again blended in a conta-blender. The blend was compressed using a suitable tooling to obtain tablets. The tablets were then coated with a dispersion of Opadry® in water.

While the present invention has been described in terms of its specific embodiments, certain modifications and equivalents will be apparent to those skilled in the art and are intended to be included within the scope of the present invention.

1. An oral pharmaceutical composition comprising:
a) ospemifene or a pharmaceutically acceptable salt thereof; and
b) one or more pharmaceutically acceptable excipients,
wherein the composition is devoid of any granulates and releases at least about 80% of ospemifene or a pharmaceutically acceptable salt thereof in about 30 minutes.

2. The pharmaceutical composition according to claim 1, wherein the composition is prepared by a direct compression process.

3. The pharmaceutical composition according to claim 1, wherein the composition comprises about 30 mg to about 90 mg of ospemifene.

4. The pharmaceutical composition according to claim 1, wherein 50% of ospemifene has a particle size of not more than about 15 microns.

5. The pharmaceutical composition according to claim 1, wherein the composition is in the form of a tablet, a capsule, pellets or powder.

6. The pharmaceutical composition according to claim 1, wherein the pharmaceutically acceptable excipients comprise one or more binders, fillers, disintegrants, lubricants, glidants, anti-tacking agents, or a combination thereof.

7. The pharmaceutical composition according to claim 1 further comprising one or more enhancing agents.

8. The pharmaceutical composition according to claim 7, wherein the enhancing agents comprise glycerol-polyethylene glycol oxystearates, PEG-glyceryl palmitostearate, sodium lauryl sulfate, polyoxyethylene sorbitan monooleate, benzyl alcohol, sorbitan monolaurate, Poloxamer, polyethylene glycols, polyvinylpyrrolidones, polyvinylalcohols, polyalcohols, oleic acid, glyceryl monooleate, sodium benzoate, cetyl alcohol, crospovidone, and sodium starch glycolate.

9. The pharmaceutical composition according to claim 1, prepared by a process comprising:
(a) mixing ospemifene with one or more enhancing agents and one or more pharmaceutically acceptable excipients;
(b) blending the mixture;
(c) compressing the resulting mixture into tablets; and
(d) optionally coating the tablets.
